(19) United States
(12) Patent Application Publication
(10) Pub. No.: US 2018/0078598 A1
(43) Pub. Date: Mar. 22, 2018

(54) DIETARY SUPPLEMENTS CONTAINING DEHYDRATED STRAWBERRY

(71) Applicant: Dan Legard, Watsonville, CA (US)

(72) Inventor: Dan Legard, Watsonville, CA (US)

(21) Appl. No.: 15/710,709

(22) Filed: Sep. 20, 2017

Related U.S. Application Data
(60) Provisional application No. 62/397,837, filed on Sep. 21, 2016.

(51) Int. Cl. A61K 36/73; A23L 19/00; A23L 33/105

(52) U.S. Cl. CPC A61K 36/73; A23L 19/01; A23V 2300/10; A23V 2002/00; A23L 33/105

(57) ABSTRACT

Compositions and processes are disclosed for a capsule, tablet, or pill comprised of powdered strawberries. Fresh strawberries are washed, de-calyxed, and then processed into a dry pulp. The dry pulp may be pulverized to a desired granularity. In some embodiments, the pulp may be filtered to remove the seeds, prior to drying. In some embodiments the fruit may be dried by way of freeze drying, hot air drying, or spray-dried. Additional vitamins and minerals may be included with the powdered strawberries to impart further health benefits to the composition. An advantage of this composition is that it serves several functions, including providing a good source of antioxidants, phytochemicals, and flavonoids. As such, the composition may be useful for mitigating various types of cancer, heart disease, high cholesterol, high blood pressure, cataracts, arthritis, skin damage from UV rays, anti-aging, and the like.

### CROSS-REFERENCE

This application claims the benefit of U.S. Provisional Patent Application No. 62/397,837, filed Sep. 21, 2016, titled, “DIETARY SUPPLEMENTS CONTAINING DEHYDRATED STRAWBERRY,” which is hereby incorporated by reference herein in its entirety.

### FIELD OF THE INVENTION

The field of the present disclosure generally relates to botanical and nutritional food products and dietary supplements. More particularly, the field of the present disclosure relates to compositions and processes for preparing dietary supplements comprising strawberry fruit powder and other beneficial ingredients and delivering the supplement in a capsule, tablet or pill form.

### BACKGROUND

Second only to grapes, strawberries are among the largest non-citrus fruit specialty crop in the United States, with 2014 production values at $2.9bn (USDA National Agricultural Statistics Service (NASS), 2015). Strawberry production involves repeated fruit bearing and multiple harvests per season, making strawberries the highest tonnage per acre (25 tons/acre) crop among all fruits (NASS 2012). In 2014, the average grower price for fresh strawberries was $107/hundredweight, comprising an all-time high, and an increase of 11% from 2013. Prices for processing strawberries are consistently lower than fresh market strawberries, with farmers receiving an average of $42.60/hundredweight in 2014, an increase of 22% from 2013 (NASS, 2015). Strawberries are consumed fresh, as well as processed into many foods, such as ice cream, yogurt, juices, jams, jellies, baked goods, and the like.

Strawberries are well known for being a “superfood” as they are nutrient rich and are widely considered to be a good source of antioxidants, phytochemicals, and flavonoids such as Vitamin C, ellagic acid, and lutein, as well as being a source for potassium, Vitamin B, and other essential nutrients. Such nutrients are widely accepted as having a variety of benefits including the mitigation of various types of cancer, heart disease, high cholesterol, high blood pressure, cataracts, arthritis, skin damage from UV rays, anti-aging, and the like.

The human physiological need for vitamins has been well established. Regular dietary consumption of vitamins is essential to good health. Various organizations and government agencies have published recommended quantities for vitamin consumption. One well known standard in the United States is the U.S. Recommended Daily Allowance (RDA) which recommends quantities for the daily intake of vitamins. While the need for vitamins is well known, the average person typically fails to consume the recommended daily intake of vitamins through a normal diet. Often this is due to a busy work schedule that encourages the consumption of “fast food” that is high in fat and sugar content. For others, food rich in vitamin content may just not be readily available, especially in remote areas with limited infrastructure. For a variety of reasons, many people do not consume enough vitamins on a daily basis.

Vitamin supplements have become common and are distributed in a variety of forms. Pills, capsules, elixirs, tablets and other forms abound on the market as a means for obtaining a proper daily vitamin intake. For some, this is an acceptable source of vitamins; however, many people cannot consume vitamins in these forms. Children and the elderly are especially averse to consumption of these products. This may be due to an intolerance to the supplements in a refined form, sensitive gag reflexes, or a strong distaste for the product's form or taste. Regardless of the reason, many people find concentrated vitamin supplements unpalatable and unacceptable as a source of daily vitamin intake.

Natural foods are increasingly popular among health-conscious consumers. Vitamins and other nutrients obtainable by way of naturally occurring sources are now in high demand. “Natural” vitamins generally are found in a product in its natural state without vitamin supplement or vitamin “fortification”. Because many fruits have high vitamin content, fruit flavors are often associated with healthy vitamin-rich products.

What is needed, therefore, is a composition comprising a natural source of essential nutrients that may be transported and stored over a prolonged period of time and consumed in a variety of convenient forms such as capsules, tablets or pills that may be consumed quickly and easily while “on the go”.

### SUMMARY

In an aspect of the invention a solid dietary supplement is provided comprising, a portion of dehydrated strawberry fruit powder having a granularity and purity suitable for human consumption. In an aspect of the invention, a solid dietary supplement may be configured to be disposed within a capsule. In an aspect of the invention, a solid dietary supplement may be configured to be provided in a tablet or pill form.

In an aspect of the invention a powdered dietary supplement in capsule, pill or tablet form is provided comprising, a first portion comprising a dehydrated strawberry fruit powder having a granularity and purity suitable for human consumption; and a second portion comprising supplemental ingredients. In an aspect of the invention the powdered dietary supplement in capsule, pill or tablet form further comprises a natural health promoting ingredient, herbal ingredient or combination thereof.

In an aspect of the invention, a method of producing a powdered dietary supplement is provided comprising, providing a strawberry, washing the strawberry, removing a calyx of the strawberry, drying the strawberry, and crushing the strawberry into a powder. In an aspect of the invention, the method further includes drying the strawberry includes any one of freeze drying, hot air drying, or spray-drying. In an aspect of the invention, the method of crushing the strawberry further comprises adding one or more additional nutrients, wherein the one or more additional nutrients are selected from a group consisting of, vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or a combination thereof. In an aspect of the invention, the method further comprises forming the powder into a tablet or pill. In an aspect of the invention, the method further comprises packaging the powder into a capsule.

In an aspect of the invention, a method of producing a dietary supplement for human consumption is provided comprising, providing a strawberry, washing the strawberry, removing a calyx of the strawberry, blending the strawberry into a pulp, filtering the pulp to remove any seeds, drying the pulp, and crushing the pulp into a powder. In an aspect of the invention, the method may include wherein drying the strawberry comprises freeze drying, hot air drying or spray-drying. In an aspect of the invention, the method may include wherein crushing the strawberry further comprises adding at least one additional nutrient. In an aspect of the invention, the method may include wherein the at least one additional nutrient is comprised of vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or any combination thereof. In an aspect of the invention, the method may include forming the powder into a tablet or pill. In an aspect of the invention, the method may include dispensing the powder into a capsule.

In an aspect of the invention, a method of producing a dietary supplement for human consumption is provided comprising, providing a strawberry, washing the strawberry, removing a calyx of the strawberry, blending the strawberry into a pulp, filtering the pulp to remove any seeds, filtering the pulp to separate solids from any juice comprising the pulp, mixing a first portion of the juice with a second portion of the solids to create a mixture, drying the mixture, and crushing the mixture into a powder. In an aspect of the invention, the method may include wherein drying the mixture comprises freeze drying, hot air drying, or spray-drying. In an aspect of the invention, the method may include, wherein crushing the mixture further comprises adding one or more additional nutrients, and wherein the one or more additional nutrients are comprised of vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or any combination thereof. In an aspect of the invention, the method may include forming the powder into a tablet or pill. In an aspect of the invention, the method may include dispensing the powder into a capsule or a pill.

### DESCRIPTION OF THE DRAWINGS

The drawings refer to embodiments of the present disclosure in which:

FIG. 1 illustrates a side view of an exemplary pill with a cut-away section to illustrate a powder disposed therein.

FIG. 2 illustrates an exploded view of the exemplary pill of FIG. 1.

FIG. 3 illustrates a side view of an exemplary capsule with a cut-away section to illustrate the compressed powder and a coating disposed thereon.

FIG. 4 illustrates an exemplary process of producing a powder in accordance with some embodiments.

FIG. 5 illustrates an exemplary process of producing a powder in accordance with some embodiments.

FIG. 6 illustrates an exemplary process of producing a powder in accordance with some embodiments.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “a first compound,” may be made. However, the specific numeric reference should not be interpreted as a literal sequential order but rather interpreted that the “first compound” is different than a “second compound.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In accordance with the present disclosure, a composition for a dietary supplement comprising, a powder that includes strawberry fruit and other select ingredients. In some embodiments the strawberry fruit is comprised of fruit from the Fragaria genus. In some embodiments preferable species or hybrids of strawberry may include Fragaria×ananassa, Fragaria×bringhurstii, Fragaria×vescana, Fragaria vesca, and Fragaria chiloensis, although other species of Fragaria are contemplated.

It should be noted that the colloquially termed “fruit” of Fragaria spp. is not a botanical berry, but rather is an aggregate accessory fruit characterized by a fleshy part that is derived not from the plant's ovaries, but from the receptacle that holds the ovaries. Each apparent “seed” (achene) on the outside of the fruit is actually one of the ovaries of the flower, with a seed inside it. Thus, it will be appreciated that the term “fruit” or “strawberry fruit”, referred to herein, refers to the aforementioned aggregate accessory fruit.

The strawberry fruit is widely appreciated for its characteristic aroma, bright red color, juicy texture, and sweetness as well as its nutritional value. Strawberries are considered an excellent source of vitamin C, a good source of manganese, and provide several other vitamins and dietary minerals such as Thiamine (B1), Riboflavin (B2), Niacin (B3), Pantothenic acid (B5), Vitamin B6, Folate (B9), Vitamin C, Vitamin E, Vitamin K, Calcium, Iron, Magnesium, Manganese, Phosphorus, Potassium, and Zinc.

Strawberries are consumed in large quantities, either fresh or in such prepared foods as preserves, fruit juice, pies, ice creams, milkshakes, chocolates, and the like. Artificial strawberry flavorings and aromas are also widely used in many products, such as lip gloss, candy, hand sanitizers, perfume, and many others. Strawberries are well known as being a “superfood” as they are nutrient rich and are widely considered to be good source of Vitamin C, manganese, ellagic acid, lutein, zeathancins and other phytochemicals, flavonoids, and antioxidants, as well as a source for potassium, Vitamin B, and other essential nutrients.

As a result, strawberries are recognized as providing a variety of health benefits including mitigating or preventing various forms of cancer, cardiovascular disease, and high cholesterol. The antioxidants found in strawberries help reduce the occurrence of cataracts, arthritis, and skin damage from UV rays. Phytochemicals present in strawberries have anti-inflammatory or anticancer properties. Further, epidemiological studies have associated strawberry consumption with lower rates of hypertension, inflammation, cancer, and death from cardiovascular diseases.

In general, during preparation of a powder comprising the strawberry fruit, the fruit may be washed and the calyx of the strawberry may be removed. It is contemplated that the calyx maybe be removed by way of a process described in U.S. Pat. No. 9,364,020 titled, “Automated Fruit and Vegetable Calyx or Stem Removal Machine” and granted Jun. 14, 2016, which is incorporated by reference herein. The processed fruit may then be dried. In some embodiments, the fruit may be “freeze dried” whereby the fruit is frozen and then dehydrated in a vacuum. In some embodiments the fruit may be dried by way of hot air drying wherein fruit or fruit pulp is placed on a conveyor belt and exposed to extremely hot air. Once dried, the fruit may then be crushed or pulverized into a suitable powder of desired granularity by any number of processes that are known in the art. In some embodiments, additional ingredients, such as, by way of non-limiting example, vitamins, minerals, herbs or non-active ingredients, may be added to the powder, as will be described herein. In some embodiments, a pharmaceutically acceptable carrier, excipient, buffer or stabilizer may be added to the powder, as will be described herein. In some embodiments the powder may be disposed within a capsule, or compressed into a tablet or pill.

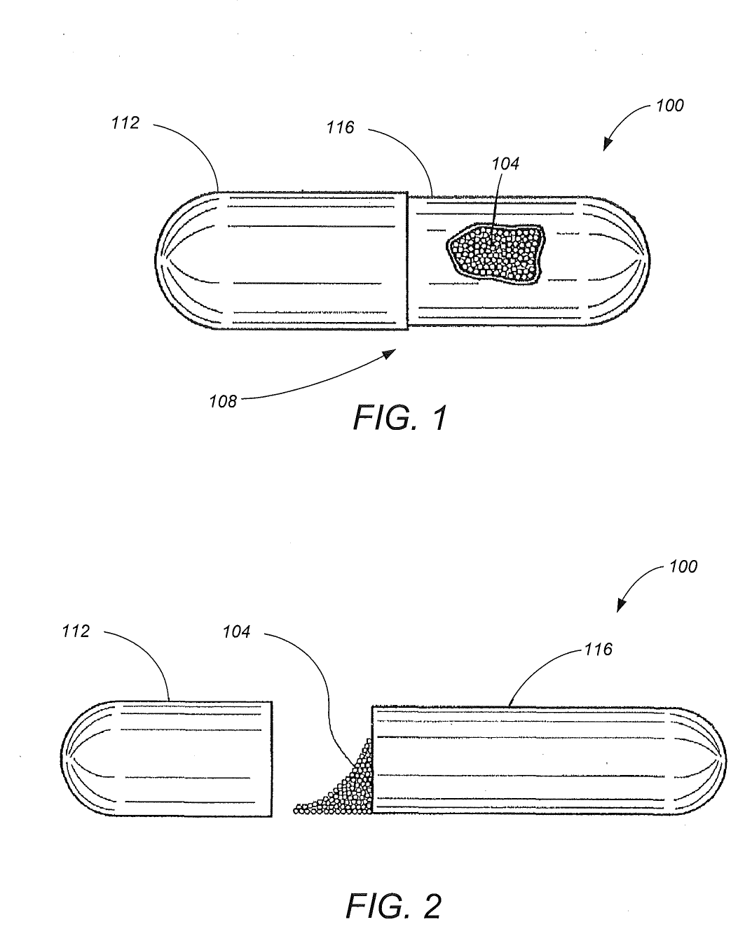

FIG. 1 illustrates an exemplary capsule 100 with a powder 104 disposed therein. The capsule 100 includes a capsule casing 108, the capsule casing 108 may comprise of one or more portions. By way of a non-limiting example, FIG. 1 illustrates the capsule casing 108 comprising a first portion 112 and a second portion 116. In some embodiments, the capsule casing 108 may be soluble after ingestion to release the powder disposed therein. In some embodiments, the capsule casing 108 may be made of a synthetic, vegetable, or animal based product, such as, by way of non-limiting example, cellulose polymers, hypromellose, methylcellulose, gelatin, spun proteins, or the like. In some embodiments the capsule casing 108 may be flavored.

FIG. 2 illustrates an exploded view of the capsule 100 with the first portion 112 of capsule casing 108 separated from the second portion 116 of capsule casing 108. FIG. 2 also illustrates the powder 104 contained within capsule casing 108. FIGS. 1 and 2 illustrate how the second portion 116 of capsule casing 108 fits within the first portion 112 of capsule casing 108 so as to create a snug fit and contain the powder 104 therein. It will be appreciated that other processes of coupling, sealing or connecting the first and second portions 112, 116 fall within the scope of the present invention.

In some embodiments, the capsule 100 may range in size between substantially size 13 and size 5, or between size 00 and size 2, or be substantially size 00. In some embodiments, the capsule 100 may contain between substantially 3200 milligrams (mg) and 60 mg of powder, or between 950 mg and 165 mg of powder, or substantially 700 mg of powder. In an exemplary embodiment, the powder 104 comprises between 1% and 99% of dried granulated strawberries, or between 60% to 90% of dried granulated strawberries, or substantially 85% of dried granulated strawberries.

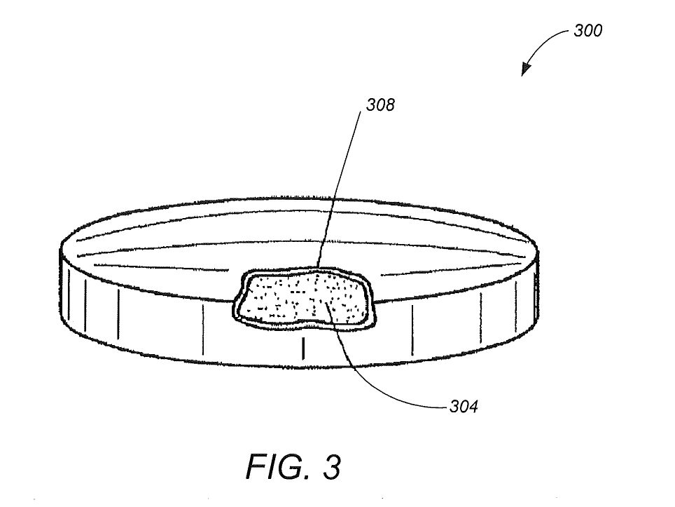

FIG. 3 illustrates an exemplary tablet 300. In some embodiments, powder 304 may be pressure formed to form a solid tablet 300 by using a suitable excipient or binder. Exemplary excipients may include lactose, dibasic calcium phosphate, sucrose, corn (maize) starch, microcrystalline cellulose, povidone polyvinylpyrrolidone, modified cellulose (for example hydroxypropyl methylcellulose or hydroxyethylcellulose), or the like. Suitable pressure forming processes may include wet granulation, dry granulation or other tablet forming processes that are known in the art.

In some embodiments the tablet 300 may be coated with a coating 308 in order to create a pill. Exemplary coatings may include polymer and polysaccharide based coatings, and may also include plasticizers and pigments. In some embodiments, the coating 308 may be made of a synthetic, vegetable, or animal based product, such as, by way of non-limiting example, cellulose polymers, hypromellose, methylcellulose, gelatin, spun proteins, or the like. The coating 308 may advantageously be stable and strong enough to allow the tablet to be handled during manufacture and transport prior to ingestion. The coating 308 may also prevent the tablet or pill from sticking together. In some embodiments, the coating 308 may give the tablet or pill an aesthetically pleasing finish or taste. The coating 308 may also give the tablet an extended shelf-life.

In some embodiments, the tablet 300 may range in size between substantially size T10 and size T1, or between size T8 and size T2, or substantially size T4. In some embodiments, the tablet 300 may contain between substantially 3200 milligrams (mg) and 60 mg of powder, or between 950 mg and 165 mg of powder, or substantially 700 mg of powder. In an exemplary embodiment, the powder 304 comprises between 1% and 99% of dried granulated strawberries, or between 60% to 90% of dried granulated strawberries, or substantially 85% of dried granulated strawberries.

It will be appreciated that other forms of pill, capsule or tablet will fall within the scope of the present invention, including caplets, orally disintegrating tablets (ODT), ‘chewable’ tablets, ‘gummies’, ‘softgel’ capsules, or the like. It will also be appreciated that the shape of the tablet, pill or capsule is not limited to those illustrated herein, and may include round, oblong, oval, square, rectangle, diamond, 3 to 8 sided embodiments, or the like. In some embodiments alphanumeric symbols, logos or other indicia made stamped or etched on to the surface of the tablet, pill or capsule.

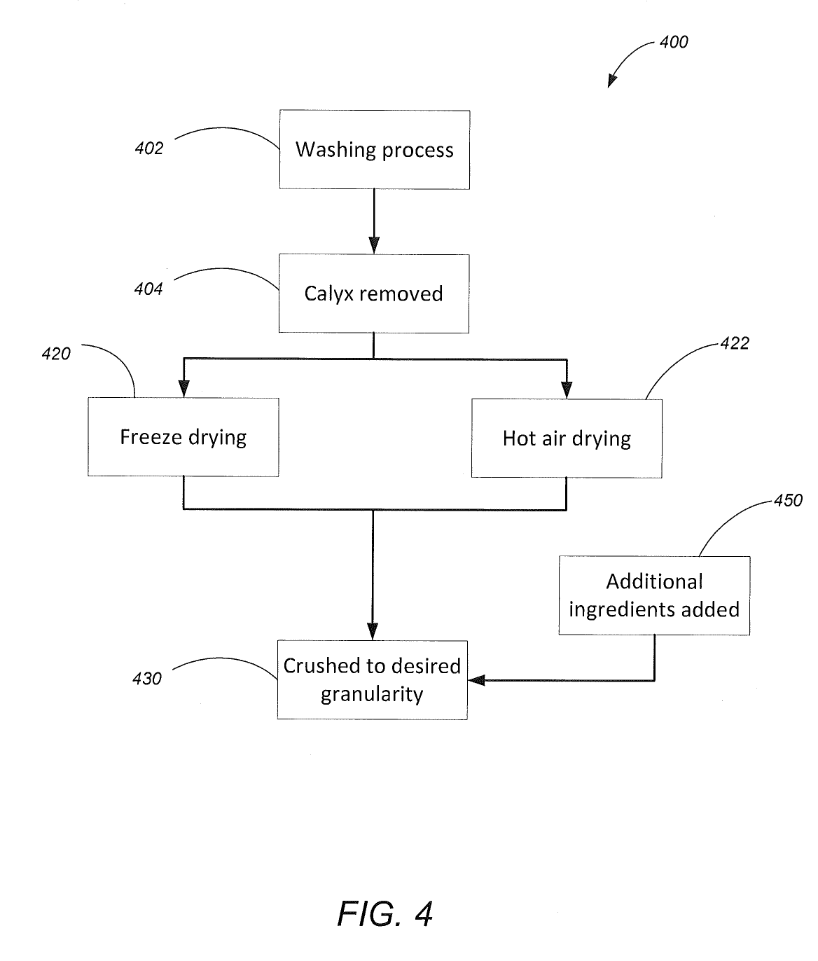

FIG. 4 illustrates a process 400 of preparing a powder comprising the strawberry fruit, in accordance with some embodiments. The fruit may be washed 402 and the calyx of the strawberry may be removed 404, as previously described herein. The processed fruit may then be dried. In some embodiments, the fruit may be freeze dried 420 whereby the fruit is frozen and then dehydrated in a vacuum. In some embodiments the fruit may be dried by way of hot air drying 422 wherein fruit is placed on a conveyor belt and exposed to extremely hot air. Once dried, the fruit may then be crushed or pulverized 430 into a suitable powder of desired granularity by any number of processes that are known in the art. In some embodiments, additional ingredients 450, such as, by way of non-limiting example, vitamins, minerals, herbs or non-active ingredients, may be added to the powder 430, as will be described herein.

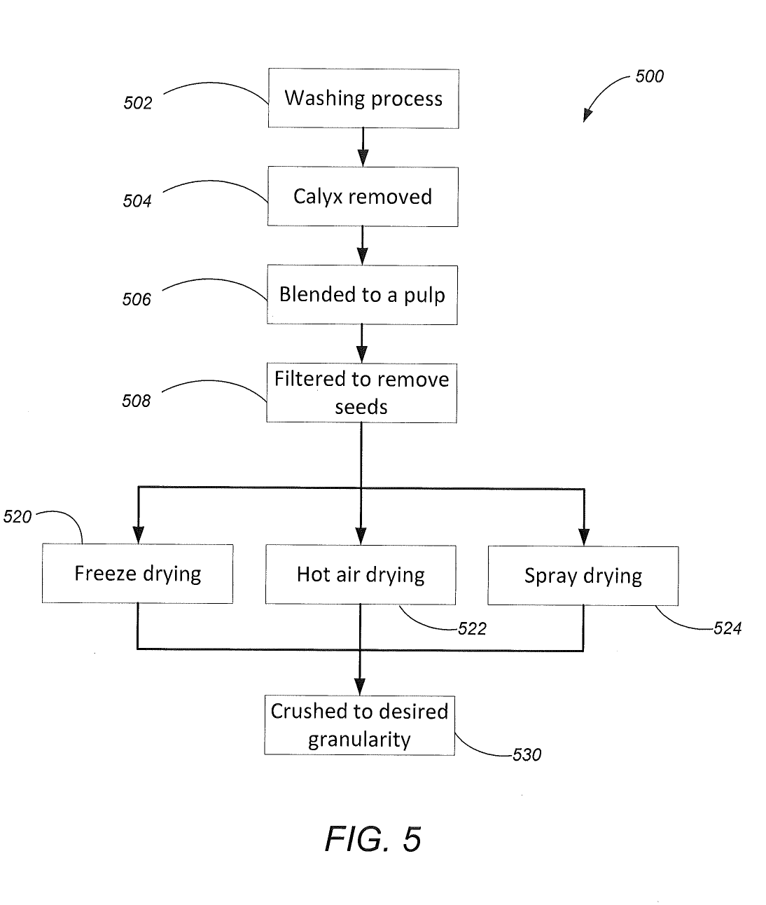

FIG. 5 illustrates a process 500 of preparing a powder comprising the strawberry fruit, in accordance with some embodiments. In some embodiments, the fruit may be washed 502 and the calyxes may be removed 504, as described herein, before the fruit are blended into a pulp 506, The pulp may be filtered to remove seeds 508 and then dried by way of any suitable drying processes, including freeze drying 520 or hot air drying 522. In some embodiments the pulp may be spray-dried 524, wherein the pulp is sent to an atomizer which uses a nozzle or centrifugal rotating wheel to atomize the pulp into a spray. Droplets created by the atomizer contain both water and strawberry fruit solids. This atomized spray is sent to a drying chamber with a controlled temperature and air flow. The atomized spray is kept in contact with the heated air until a desired moisture content is achieved, thereby yielding dried fruit that may then be crushed or pulverized to a desired granularity 530.

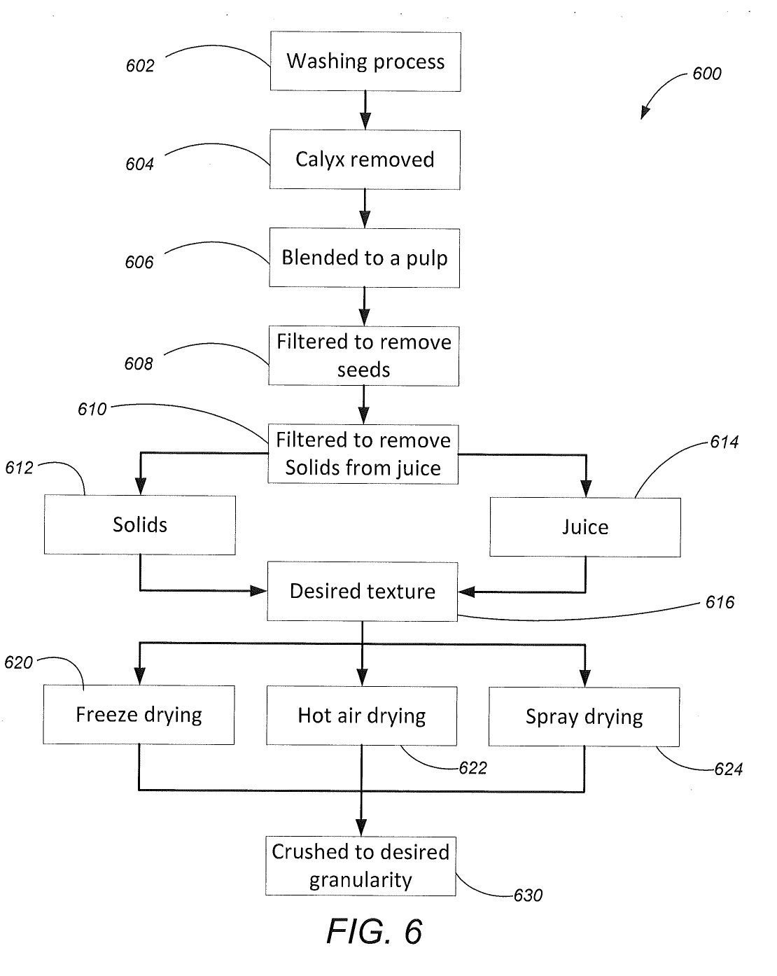

FIG. 6 illustrates a process 600 of preparing a powder comprising the strawberry fruit, in accordance with some embodiments. The fruit may be washed 602, the calyx removed 604, and blended into a pulp 606, as described herein. After filtering to remove seeds 608, the pulp may be passed through a 0.2 micron micro-filtration system 610 so as to remove the solids 612 from the juice 614. It is contemplated that the consistency of the remaining solids 612 may be adjusted to a desired texture 616 by reintroducing the juice 614, adding an alternative solvent suitable for human consumption (e.g. water or similar suitable solvent), or a combination thereof. Once a desired consistency is achieved, the pulp may be then dried by way of any of freeze drying 620, hot air drying 622 or spray-drying 624, as described herein. The resulting dried fruit may then be crushed or pulverized to a desired granularity 630.

In some embodiments, the juice extracted from the strawberry pulp may be purified so as to form a concentrated extract or syrup. The purification may be performed either on the pure juice of the strawberry or after mixing the juice with other ingredients. The concentrated extract may then be dried to a powder by way of freeze drying, hot air drying, or spray drying, as described herein. In some embodiments wherein more fiber or texture is desired, a relatively coarser filter may be used in lieu of the above mentioned 0.2 micron micro-filtration system. Further, in some embodiments, fiber and texture producing ingredients may be added so as to produce a desired powder texture and consistency.

In some embodiments, additional ingredients, such as, by way of non-limiting example, vitamins, minerals, herbs or non-active ingredients, may be added to the powder or to the pulp, juice or mixture prior to the drying process. It is contemplated that vitamins or minerals that may be added include vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, as well as any combination thereof.

In some embodiments, a pharmaceutically acceptable carrier, excipient, buffer or stabilizer may be added to the powder, pulp, juice or mixture prior to the drying process. It is contemplated that suitable pharmaceutically acceptable carriers include essentially chemically inert and nontoxic materials that do not interfere with the effectiveness of the biological activity of the composition. Examples of suitable pharmaceutical carriers include, but are not limited to, water, saline solutions, glycerol solutions, ethanol, N-(1 (2,3-dioleyloxy)propyl)N,N,N-trimethylammonium chloride (DOTMA), diolesylphosphotidyl-ethanolamine (DOPE), and liposomes. Further, in some embodiments, flavor and/or color additives may be included in the powdered composition.

In some embodiments, as previously described herein, the powdered composition may be pressed into tablets, or dispensed in capsules or pills, as desired. In some embodiments the powdered composition may be used directly as an ingredient in food or drink products, including but not limited to soups, drinks, sauces, salad dressings, breads, pastas and any other foods into which the powdered composition may be incorporated.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims

What is claimed:

1. A solid dietary supplement, comprising:
a portion of dehydrated strawberry fruit powder having a granularity and purity suitable for human consumption.

2. The supplement of claim 1, wherein the solid dietary supplement is configured to be disposed within a capsule.

3. The supplement of claim 1, wherein the solid supplement is configured to be provided in a tablet or pill form.

4. A powdered dietary supplement in capsule, pill or tablet form, comprising:
a first portion comprising a dehydrated strawberry fruit powder having a granularity and purity suitable for human consumption; and
a second portion comprising supplemental ingredients.

5. The supplement of claim 4, wherein the second portion comprises a natural health promoting ingredient, herbal ingredient or combination thereof.

6. A method of producing a powdered dietary supplement, comprising:
providing a strawberry;
washing the strawberry;
removing a calyx of the strawberry;
drying the strawberry; and
crushing the strawberry into a powder.

7. The method of claim 6, wherein drying the strawberry includes any one of freeze drying, hot air drying, or spray-drying.

8. The method of claim 6, wherein crushing the strawberry further comprises adding one or more additional nutrients.

9. The method of claim 8, wherein the one or more additional nutrients are selected from a group consisting of, vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or a combination thereof.

10. The method of claim 6, further comprising forming the powder into a tablet or pill.

11. The method of claim 6, further comprising packaging the powder into a capsule.

12. A method of producing a dietary supplement for human consumption comprising:
providing a strawberry;
washing the strawberry;
removing a calyx of the strawberry;
blending the strawberry into a pulp;
filtering the pulp to remove any seeds;
drying the pulp; and
crushing the pulp into a powder.

13. The method of claim 12, wherein drying the strawberry comprises freeze drying, hot air drying or spray-drying.

14. The method of claim 12, wherein crushing the strawberry further comprises adding at least one additional nutrient.

15. The method of claim 14, wherein the at least one additional nutrient is comprised of vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or any combination thereof.

16. The method of claim 12, further comprising forming the powder into a tablet or pill.

17. The method of claim 12, further comprising dispensing the powder into a capsule.

18. A method of producing a dietary supplement for human consumption comprising:
providing a strawberry;
washing the strawberry;
removing a calyx of the strawberry;
blending the strawberry into a pulp;
filtering the pulp to remove any seeds;
filtering the pulp to separate solids from any juice comprising the pulp;
mixing a first portion of the juice with a second portion of the solids to create a mixture;
drying the mixture; and
crushing the mixture into a powder.

19. The method of claim 18, wherein drying the mixture comprises freeze drying, hot air drying, or spray-drying.

20. The method of claim 18, wherein crushing the mixture further comprises adding one or more additional nutrients.

21. The method of claim 20, wherein the one or more additional nutrients are comprised of vitamin A, vitamin B1, vitamin B2, vitamin B3, vitamin B5, vitamin B6, vitamin B9, vitamin B12, vitamin C, vitamin D, vitamin E, vitamin K, calcium, chromium, coenzyme Q10, folate, iodine, iron, magnesium, potassium, riboflavin, selenium, thiamin, zinc, or any combination thereof.

22. The method of claim 18, further comprising forming the powder into a tablet or pill.

23. The method of claim 18, further comprising dispensing the powder into a capsule or a pill.
